(19) United States
(12) Patent Application Publication
(10) Pub. No.: US 2005/0059933 A1
(43) Pub. Date: Mar. 17, 2005

(54) Safety intravenous (IV) catheter assembly

(76) Inventor: Carolyn M. Johnson, St. Peters, MO (US)

Correspondence Address: LAW OFFICES OF ERIC KARICH, 2807 ST. MARK DR., MANSFIELD, TX, 76063, US

(21) Appl. No.: 10/974,636

(22) Filed: Oct. 27, 2004

Related U.S. Application Data
(63) Continuation-in-part of application No. 10/329,844, filed on Dec. 27, 2002.

(51) Int. Cl. A61M 5/178

(52) U.S. Cl. 604/164.01

(57) ABSTRACT

A catheter assembly is disclosed including a needle, a container, a tubular body, and a pair of tabs. The container includes a chamber adapted to receive the needle and a mechanism adjacent an opening. The mechanism is adapted for engaging an end of the needle and for moving the needle into the chamber when activated. The tubular body has opposed catheter and connection ends. The catheter end is adapted for insertion into a vein and has a channel adapted to receive the needle. The connection end is removably attached to the opening of the container and is adapted for connection to a fluid transfer device. The pair of tabs extend outwardly from opposite sides of the body adjacent the catheter.

### CROSS-REFERENCE TO RELATED APPLICATIONS

This application for a utility patent is a continuation-in-part of a previously filed utility patent, now abandoned, having the application Ser. No. 10/329,844, filed Dec. 27, 2002. This application is incorporated herein by reference in its entirety.

### STATEMENT REGARDING FEDERALLY SPONSORED RESEARCH

Not Applicable

### BACKGROUND OF THE INVENTION

1. Field of the Invention

This invention relates generally to a medical apparatus, and more particularly to intravenous (IV) delivery systems.

2. Description of Related Art

Conventional intravenous (IV) delivery systems have several significant problems that put health care workers at risk. In a typical IV catheter insertion procedure, one end of an IV catheter, with a sharp end of needle extending therethrough, is usually pushed through the skin of a patient's arm and into a vein. After the end of the catheter is in the vein, the health care worker typically applies pressure to the insertion site with one hand, and removes and disposes of the needle and subsequently applies a piece of tape over the IV catheter at the insertion site with the other hand. The piece of tape is used to anchor the IV catheter in place.

Removing and properly. disposing of the contaminated needle with one hand while remaining focused on the injection site is often difficult, and needle sticks are all too common. Further, when the needle is removed from the catheter, or while the piece of tape is being applied to anchor the IV catheter, blood frequently exits the catheter and contacts the health care worker.

A needle stick and/or other contact with the patient's blood subjects the health care worker to contagious diseases such as the human immunodeficiency virus (HIV), the most severe manifestation of HIV known as the acquired immunodeficiency syndrome (AIDS), and hepatitis.

It would thus be advantageous to have an IV catheter assembly with components designed to be operated with one hand and that shields a user from a contaminated needle following use of the needle to insert a catheter into a vein.

### SUMMARY OF THE INVENTION

A catheter assembly is disclosed including a needle, a container, a tubular body, and a pair of tabs. The container includes a chamber adapted to receive the needle and a mechanism adjacent an opening. The mechanism is adapted for engaging an end of the needle and for moving the needle into the chamber when activated. The tubular body has opposed catheter and connection ends. The catheter end is adapted for insertion into a vein and has a channel adapted to receive the needle. The connection end is removably attached to the opening of the container and is adapted for connection to a fluid transfer device. The pair of tabs extend outwardly from opposite sides of the body adjacent the catheter end, and one side of each of the tabs has a layer of an adhesive material applied thereto.

Other features and advantages of the present invention will become apparent from the following more detailed description, taken in conjunction with the accompanying drawings, which illustrate, by way of example, the principles of the invention.

### BRIEF DESCRIPTION OF THE DRAWING

The accompanying drawings illustrate the present invention. In such drawings:

FIG. 1 is a perspective view of one embodiment of a safety intravenous (IV) catheter assembly including a tubular body having a catheter end; and

FIG. 2 is a perspective view of the catheter assembly of FIG. 1 wherein the catheter end of the body has been inserted into a vein of an arm of a patient at an insertion site.

### DETAILED DESCRIPTION OF THE INVENTION

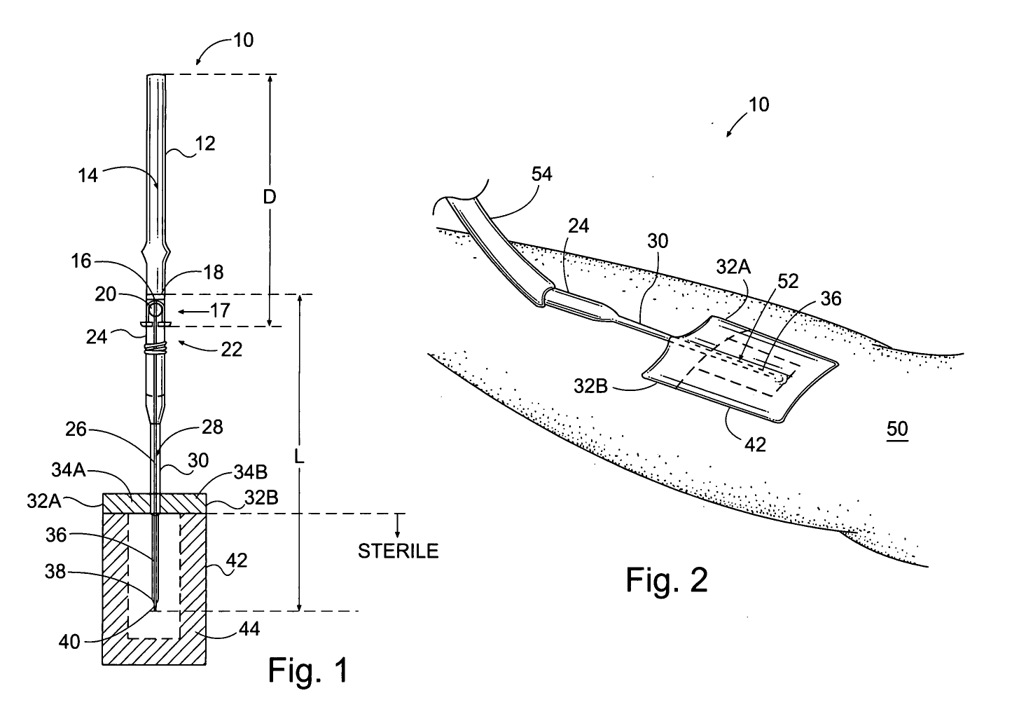

FIG. 1 is a perspective view of one embodiment of a safety intravenous (IV) catheter assembly 10 including a needle 26 extending through a channel 28 within a tubular body 30. The tubular body 30 has a catheter end 36 and an opposed connection end 24. In general, the catheter end 36 is adapted for insertion into a vein (e.g., of a human patient), and the connection end 24 is adapted for connection to a fluid transfer device. The channel 28 extends through an axial center of the tubular body 30, and is generally adapted to receive the needle 26 and to act as a conduit for fluid flow between the catheter end 36 and the connection end 24.

The needle 26 has a sharp end 40 and an opposite end 16. In FIG. 1, the sharp end 40 of the needle 26 extends out of a tip 38 of the catheter end 36. The opposite end 16 of the needle 26 extends out of the connection end 24 of the body 30 and into an opening 22 of a container 12. The needle 26 is preferably sterile. Further, the catheter end 36 of the body 30 is also preferably sterile as indicated in FIG. 1. In the configuration of FIG. 1, the sharp end 40 of the needle 26 and the catheter end 36 of the body 30 are ready for insertion into a vein.

In general, the connection end 24 of the body 30 is removably attached to the opening 22 of the container 12. In the embodiment of FIG. 1, the opening 22 of the container 12 is attached to the connection end 24 of the body 30 such that the container 12 can be “snapped off” of the connection end 24. After the container 12 is removed from the connection end 24 of the body 30, a fluid transfer device may be connected to the connection end 24.

The opening 22 of the container 12 leads into a chamber 14. In general, the chamber 14 of the container 12 is adapted to receive the needle 26. More specifically, the needle 26 has a length dimension “L” between the sharp end 40 and the opposite end 16, and the chamber 14 has a dimension “D” that is greater than the length dimension L of the needle 26. In the embodiment of FIG. 1, the chamber 14 is cylindrical and coaxial with the channel 28 of the tubular body 30.

A mechanism 17 is positioned in the container 12 adjacent the opening 22. In general, the mechanism 17 is adapted for engaging the end 16 of the needle 26 and for moving the needle 26 into the chamber 14 of the container 12 when the mechanism 17 is activated. In the embodiment of FIG. 1, the mechanism 17 includes a compressed spring 18 and a button 20 coupled to the compressed spring 18. Pressing the button 20 activates the mechanism 17, releases the compressed spring 18 such that the spring 18 moves the needle 26 from the body 30 into the chamber 14 of the container 12. The container 12 is preferably a disposable plastic container.

After a user properly inserts the sharp end 40 of the needle 26 and the catheter end 36 of the body 30 into a vein, the user presses the button 20 to activate the mechanism 17. It is noted that the pressing of the button 20 to activate the mechanism 17 is expectedly easily accomplished with one hand. When the mechanism 17 is activated, the compressed spring 18 spring moves the needle 26 from the body 30 into the chamber 14 of the container 12, leaving the catheter end 36 of the body 30 in the vein. The user can then remove the container 12 from the connection end 24 of the body 30 (e.g., by snapping the container 12 off of the connection end 24), and properly dispose of the container 12 (with the contaminated needle 26 inside). During this procedure, the user is advantageously shielded from, and cannot be stuck by, the contaminated needle 26.

In the embodiment of FIG. 1, the body 30 is substantially transparent such that when the user is inserting the sharp end 40 of the needle 26 and the catheter end 36 of the body 30 into a vein, the user can see a “flash” of blood through the body 30 indicating successful insertion into a vein. The body 30 is preferably made of a clear plastic material.

In the embodiment of FIG. 1, a pair of tabs 32A and 32B extend outwardly from opposite sides of the body 30 adjacent the catheter end 36. In general, the tabs 32A and 32B are flexible, yet rigid enough that they may be used to guide the catheter end 36 of the body 30 as the sharp end 40 of the needle 26 and the catheter end 36 are inserted into a vein.

In the embodiment of FIG. 1, a visible sides of the tabs 32A and 32B have respective layers 34A and 34B of an adhesive material applied thereto. The adhesive layers 34A and 34B may be used to help anchor the body 30 in place after the catheter end 36 of the body 30 is inserted into the vein. For example, the adhesive layers 34A and 34B may initially have a plastic cover sheet extending over them. After the catheter end 36 of the body 30 is inserted into the vein, the plastic cover sheet may be removed, and the adhesive layers 34A and 34B may be adhered to the patient's skin about the body 30. The removal of such a plastic cover sheet is expectedly easily accomplished with one hand.

In the embodiment of FIG. 1, the catheter assembly 10 includes an optional dressing 42 extending from the pair of adhesive tabs 32A and 32B toward the catheter end 36 of the body 30. In general, the dressing 42 is dimensioned to extend over the catheter end 36. In the embodiment of FIG. 1, the dressing 42 extends over the tip 38 of the catheter end 36. The dressing 42 has a layer 44 of an adhesive material applied about a perimeter of one side (i.e., the visible side in FIG. 1). When the catheter end 36 of the body 30 has been inserted into a vein at an insertion site, the adhesive layer 44 about the perimeter may be used to anchor the dressing 42 such that the dressing 42 covers the insertion site. The adhesive layer 44 may, for example, be adhered to the patient's skin about the insertion site such that the dressing 42 covers the insertion site. (See FIG. 2). As indicated in FIG. 1, the dressing 42 is preferably sterile.

For example, the adhesive layer 44 about the perimeter of the dressing 42 may initially have a plastic cover sheet over it. After the catheter end 36 of the body 30 is inserted into the vein, and the body 30 has been anchored via the adhesive layers 34A and 34B of the respective tabs 32A and 32B, the plastic cover sheet may be removed, and the adhesive layer 44 may be adhered about the insertion site such that the dressing 42 covers the insertion site.

FIG. 2 is a perspective view of the catheter assembly 10 of FIG. 1 wherein the catheter end 36 of the body 30 has been inserted into a vein of an arm 50 of a patient at an insertion site 52. In the embodiment of FIG. 2, the adhesive layers 34A and 34B (FIG. 1) of the respective tabs 32A and 32B have been adhered to the patient's skin, thereby anchoring the body 30. The adhesive layer 44 (FIG. 1) about the perimeter of the dressing 42 has been adhered to the patient's skin about the insertion site 52 such that the dressing 42 forms a protective cover over the insertion site 52.

In FIG. 2, an end of an IV tube 54 is shown attached to the connection end 24 of the body 30. The IV tube 54 is, in general, a fluid transfer device, and a fluid may be delivered to the patient via the IV tube 54 and the body 30.

While the invention has been described with reference to at least one preferred embodiment, it is to be clearly understood by those skilled in the art that the invention is not limited thereto. Rather, the scope of the invention is to be interpreted only in conjunction with the appended claims.

1. A catheter assembly, comprising:
a needle;
a container comprising a chamber adapted to receive the needle and a mechanism adjacent an opening adapted for engaging an end of the needle and for moving the needle into the chamber when the mechanism is activated;
a tubular body having opposed catheter and connection ends, wherein the catheter end is adapted for insertion into a vein and has a channel adapted to receive the needle, and wherein the connection end is removably attached to the opening of the container and is adapted for connection to a fluid transfer device; and
a pair of tabs extending outwardly from opposite sides of the body adjacent the catheter end, wherein one side of each of the tabs has a layer of an adhesive material applied thereto.

2. The catheter assembly as recited in claim 1, wherein the needle is positioned within the body such that a sharp end of the needle exits a tip of the catheter end, and an opposite end of the needle extends into the opening of the container and is engaged by the mechanism of the container.

3. The catheter assembly as recited in claim 1, wherein the mechanism of the container comprises a compressed spring and a button coupled to the compressed spring, wherein pressing the button releases the compressed spring such that the spring moves the needle from the body into the chamber of the container.

4. The catheter assembly as recited in claim 1, wherein the opening of the container is attached to the connection end of the body such that the container can be snapped off of the connection end.

5. The catheter assembly as recited in claim 1, wherein after the container is removed from the connection end of the body, a fluid transfer device may be connected to the connection end.

6. The catheter assembly as recited in claim 1, wherein the body is substantially transparent.

7. The catheter assembly as recited in claim 1, wherein the tabs are flexible, yet rigid enough that they may be used to guide the catheter end of the body as the catheter end is inserted into the vein.

8. The catheter assembly as recited in claim 1, wherein the layers of adhesive material on the sides of the tabs may be used to help anchor the body in place after the catheter end of the body is inserted into the vein.

9. The catheter assembly as recited in claim 1, further comprising:
a dressing extending from the pair of adhesive tabs toward the catheter end of the body, dimensioned to extend over the catheter end.

10. The catheter assembly as recited in claim 9, wherein the dressing has a layer of an adhesive material applied about a perimeter of one side.

11. The catheter assembly as recited in claim 9, wherein the dressing extends over a tip of the catheter end of the body.

12. The catheter assembly as recited in claim 9, wherein when the catheter end of the body has been inserted into a vein at an insertion site, the layer of adhesive material about the perimeter of the one side of the dressing may be used to anchor the dressing such that the dressing covers the insertion site.

13. The catheter assembly as recited in claim 9, wherein the catheter end of the body, the needle, and the dressing are sterile.

14. A catheter assembly, comprising:
a needle;
a container comprising a chamber adapted to receive the needle and a mechanism adjacent an opening adapted for engaging an end of the needle and for moving the needle into the chamber when the mechanism is activated;
a tubular body having opposed catheter and connection ends, wherein the catheter end is adapted for insertion into a vein and has a channel adapted to receive the needle, and wherein the connection end is removably attached to the opening of the container and is adapted for connection to a fluid transfer device;
a pair of tabs extending outwardly from opposite sides of the body adjacent the catheter end, wherein one side of each of the tabs has a layer of an adhesive material applied thereto; and
a dressing extending from the pair of adhesive tabs toward the catheter end of the body, dimensioned to extend over the catheter end.

15. The catheter assembly as recited in claim 14, wherein the dressing has a layer of an adhesive material applied about a perimeter of one side.

16. The catheter assembly as recited in claim 14, wherein the dressing extends over a tip of the catheter end of the body.

17. The catheter assembly as recited in claim 14, wherein after the catheter end of the body has been inserted into a vein at an insertion site, the layer of adhesive material about the perimeter of the one side of the dressing may be used to anchor the dressing such that the dressing covers the insertion site.

18. The catheter assembly as recited in claim 14, wherein the catheter end of the body, the needle, and the dressing are sterile.
